(19) United States
(12) Patent Application Publication
(10) Pub. No.: US 2009/0076508 A1
(43) Pub. Date: Mar. 19, 2009

(54) IMPLANTABLE PROSTHESIS

(75) Inventors: Hermanus Hendricus Weinans, Driebergen-Rijssenburg (NL); Johannes Albertus Nicolaas Verhaar, Barendrecht (NL); Peter Leerkamp, Boxmeer (NL); Bob Meuzelaar, Nijmegen (NL)

Correspondence Address: BRYAN CAVE POWELL GOLDSTEIN, ONE ATLANTIC CENTER FOURTEENTH FLOOR, 1201 WEST PEACHTREE STREET NW, ATLANTA, GA, 30309-3488, US

(73) Assignee: FT INNOVATIONS (FTI) B.V., Boxmeer (NL)

(21) Appl. No.: 12/092,856

(22) PCT Filed: Nov. 7, 2006
(86) PCT No.: PCT/NL2006/050281

§ 371 Date: Sep. 30, 2008

(30) Foreign Application Priority Data
Nov. 7, 2005 (NL) 1030364

(51) Int. Cl. A61F 2/30; A61F 2/32

(52) U.S. Cl. 606/62; 623/22.11

(57) ABSTRACT

An implant having a substantially solid basic structure and a porous jacket structure at least partially enclosing the basic structure for attachment of cellular tissue wherein the basic structure and the jacket structure are connected integrally to each other and the porous jacket structure is formed substantially by a structure with open pores. The disclosure also relates to a method for manufacturing such an implant.

### PRIORITY CLAIM

This patent application is a U.S. National Phase of International Patent Application No. PCT/NL2006/050281, filed Nov. 7, 2006, which claims priority to Netherlands Patent Application No. 1030364, filed Nov. 7, 2005, the disclosures of which are incorporated herein by reference in their entirety.

### FIELD

The present disclosure relates to an implantable prosthesis.

### BACKGROUND

In the case of people with a worn or damaged joint, an implant, such as, for instance, an artificial hip, can be introduced surgically. The average lifespan of these implants is finite and depends on the activities and age of the patient and the type of implant. The lifespan of an implant is usually shorter than 20 years for relatively young patients (under 60 years of age). This means that an increasingly greater number of patients must undergo a second operation which is more serious relative to the first operation, wherein the first implant is replaced by a second implant. The lifespan of the second implant is generally shorter than the lifespan of the first implant as a result of the bone loss which has occurred around the prosthesis. Due to still further bone loss, a third operation to replace a worn second implant is generally difficult, a strain for the patient and sometimes even impossible.

There is a distinction between cemented and uncemented implants. A cemented implant is fixed in the bone by means of PMMA cement. This is the oldest principle and most experience has heretofore been acquired herewith. Bone cement is subject to an ageing process and must be removed in a second operation, whereby additional bone loss occurs. An increasing amount of experience has been acquired in the last 15 years with the use of uncemented prostheses. After 15 years, the results of the uncemented prosthesis are at least equal to the cemented stems and, according to some studies, even better. A survival of more than 90% of the stems after 15 years has been reported. In the case of uncemented implants, no cement is used for the attachment. The implant is placed in the bone as closely fitting as possible. The surface of such an implant is rough and porous, whereby the bone has the tendency and the opportunity to fix itself thereon, whereby an attachment between the implant and the bone can be realized. This process can be enhanced by a bioactive coating. The most significant drawback of uncemented prostheses is, however, the occurrence of bone loss around the prosthesis. This is caused by the fact that the stiffness of the prosthesis is much greater than the stiffness of the bone. The bone will, in fact, begin to move around the prosthesis whereby detaching and bone loss generally occurs relatively quickly. In addition, the strong metal prosthesis does not transmit the forces to the bone uniformly. More particularly, in the case of an artificial hip, more force transfer takes place on the knee side than on the hip side. The bone on the hip side is thus relieved of pressure and reacts with osteoporosis. This process is usually referred to as ‘stress-shielding’. The limited lifespan of (hip) implants forms a growing social and financial-economic problem, on the one hand because such implants are applied increasingly often in younger patients and on the other because the life expectancy of older patients continues to increase and the latter group also remains increasingly active. In the medical world there is, therefore, a great need for implants with a prolonged and preferably lifelong lifespan, whereby replacement of implants can be prevented, preferably definitively. Essential for an improved implant is a relatively good force transfer between the implant and the bone, wherein bone loss caused by ‘stress-shielding’ is minimized.

### SUMMARY

The present disclosure describes several exemplary embodiments of the present invention.

One aspect of the present disclosure provides an implant, comprising a) a substantially solid basic structure; and b) a porous jacket structure at least partially enclosing the basic structure for attachment of cellular tissue, wherein the basic structure and the jacket structure are connected integrally to each other and the porous jacket structure is formed substantially by a structure with open pores.

Another aspect of the present disclosure provides a method for manufacturing an implant having a substantially solid basic structure and a porous jacket structure at least partially enclosing the basic structure for attachment of cellular tissue, wherein the basic structure and the jacket structure are connected integrally to each other and the porous jacket structure is formed substantially by a structure with open pores, comprising a) arranging at least one foam-forming mould in an implant-forming mould; b) arranging a biocompatible material in the implant-forming mould and the foam-forming mould accommodated therein; and c) removing the foam-forming mould from the implant formed during step b).

The present disclosure provides an uncemented implant which has an improved ingrowth capacity and mechanical properties, and which is, therefore, relatively durable.

The present disclosure provides an implant having a substantially solid basic structure and a porous jacket structure at least partially enclosing the basic structure for attachment of cellular tissue, wherein the basic structure and the jacket structure are connected integrally to each other and the porous jacket structure is formed substantially by a structure with open pores. The basic structure and the jacket structure, in fact, form one whole, wherein the basic structure and the jacket structure are preferably manufactured in a single production step. Application of a relatively weak separating adhesive layer (intermediate layer) can be dispensed with due to the integral construction of the implant whereby a relatively strong and, therefore, durable implant can be realized. An additional feature of the implant according to the present disclosure is that the intrinsic properties of both the basic structure and the jacket structure can be optimized independently of each other. It is, therefore, recommended to considerably reduce the stiffness of the implant at least locally relative to the stiffness of conventional, uncemented implants, wherein the jacket structure, in particular, is preferably provided with a relatively low stiffness compared to the stiffness of the basic structure. The overall stiffness of the implant is hereby no longer determined solely by the design of the implant but also by the positioning and the thickness distribution of the jacket structure whereby the stress distribution between the implant and the bone can be optimized and wherein interface stresses can be minimized and the connection is thus more durable. The fit of the implant according to the present disclosure can moreover be optimized in relatively simple manner for the specific application thereof. Such optimization thus results in an improved attachment of the bone to the implant while the overall stiffness of the implant is reduced, which results in a relatively strong, reliable and durable implant. It is noted that the present disclosure is by no means limited to hip implants. On the contrary, the present disclosure relates to implants in a general sense, which can be applied for the purpose of replacing or completing a missing or deficient body part in both humans and animals. Examples of applicable implants include, among others, a total hip prosthesis, both the femur and the acetabulum components, a total knee prosthesis, both the femur and the tibia components, shoulder prosthesis, finger prosthesis, cages (intervertebral spacers), dental implants, soft part anchors, and implants for oncology.

The porous jacket structure is formed substantially by a structure with open pores, such as a foam which is provided with open cells. Advantages of applying a foam are that foam is relatively lightweight and relatively strong and, above all, has a porous structure which corresponds substantially with the micro-structure which is present in natural spongy (cancellous) bone and, therefore, functions as a matrix for receiving cellular bone tissue. The foam furthermore provides a permeability and a relatively high specific surface area for enhancing ingrowth of new bone and thus enables an improved and durable anchoring of the implant. The porosity of the foam has a gradual progression as seen in the thickness direction. The porosity of the jacket structure preferably increases in the thickness direction, wherein a part of the jacket structure integrally connected to the basic structure has a relatively low porosity, and wherein a part of the jacket structure remote from the basic structure has a relatively high porosity. Such a gradual change in the porosity, as seen in the thickness direction, has the advantage, on the one hand, that a relatively strong implant can be provided in relatively little empty space in or just around the core of the implant and, on the other hand, that the highly porous part directed toward the bone has a relatively open structure and can deform and adjust itself relatively easily to the adjacent bone. A relatively large contact surface is moreover provided by the external relatively open jacket structure, whereby the bone (in)growth can be optimized. In particular, a part of the jacket structure remote from the basic structure preferably has a porosity similar to that of porous bone in order to enable further optimization of the bone (in)growth, thereby achieving an optimal attachment between bone and prosthesis. The jacket structure is preferably at least partly plastically deformable (at relatively high forces) whereby the stress peaks during a shock load disappear as a result of shock absorption and the shock-absorbing capacity of the implant can be increased substantially, which can considerably enhance the lifespan of the implant.

In order to be able to optimize the mutual adhesion of the jacket structure to the basic structure of the integrally constructed implant, the material composition of the basic structure and the jacket structure can be substantially similar. In this manner, a homogeneously constructed implant can be provided which is relatively strong and can be introduced in relatively durable manner in a human (or animal) body.

Although the implant according to the present disclosure can be manufactured from diverse materials, at least a part of the implant is preferably manufactured from at least one of the materials from the group consisting of a biocompatible metal, a biocompatible ceramic, a biocompatible plastic and a biocompatible material with a glass-like structure. In the case a biocompatible metal is applied, it is however also possible to envisage a metal alloy being applied. The metal or the metal alloy is preferably chosen from the group consisting of Ti, TiNb, TiV, Ta, TaNb, CoCr, CoCrMo, stainless steel, alloys and combinations thereof. Titanium and titanium alloys, such as, for instance, Ti₆Al₄V, as well as cobalt chrome alloys and stainless steel are usually recommended due to the proven biocompatibility of these materials and the processability of these materials for the purpose of being able to realize an implant with an integral construction according to the present disclosure. The biocompatible materials with a glass-like structure are usually formed by amorphous metal alloys (referred to as “bulk metallic glass alloy”). Such materials are generally stronger than steel, little susceptible to wear, harder than ceramic, but also have a relatively high elasticity.

The number of pores per inch (ppi) in the jacket structure is preferably substantially greater than 10 ppi, more preferably between 60 and 100 ppi. Jacket structures with a ppi content higher than 60 ppi are relatively open, which can facilitate bone (in)growth. The number of pores per inch in the jacket structure is preferably substantially constant. As stated in the foregoing, it is, however, also advantageous to allow the porosity of the jacket structure to progress gradually as seen in the thickness direction of the jacket structure. The porosity can be reduced by increasing the thread thickness of the porous network of the jacket structure, whereby the properties of the jacket structure can be optimized. The basic structure and the jacket structure are preferably manufactured during a single production step by means of casting of liquidized biocompatible material in a mould. To enable facilitation of the casting process, a jacket structure is preferably applied with a number of pores per inch between 30 and 45 ppi. The pore size defining the porosity, at a substantially constant ppi content, preferably lies between 100 and 1500 μm, more preferably between 200 and 500 μm. The thickness of the jacket structure can vary but preferably amounts to at least three times the pore size of the jacket structure in order to be able to realize significant bone (in)growth. More preferably, the thickness of the jacket structure lies substantially between 300 μm and 15 mm. The thickness of the jacket structure can herein vary depending on the positioning of the part of the jacket structure. It is, however, also possible to envisage the thickness of the jacket structure being substantially uniform. The Young's modulus of elasticity of the jacket structure is preferably greater than 0.5 GPa, and more preferably lies between 5 and 30 GPa. Both the compression strength and the tensile strength are preferably at least 10 MPa in order to enable a sufficiently reliable implant to be provided.

In one exemplary embodiment, the jacket structure is provided with at least one of the additives from the group consisting of bone growth-stimulating agents, angiogenesis-stimulating factors, antibacterial agents and inflammation inhibitors. In order to improve the biocompatibility of the jacket structure, the pores of the jacket structure can be provided with material containing calcium and/or phosphate. Examples hereof are hydroxyapatite (HA), fluorapatite, tricalcium phosphate (TCP) and tetracalcium phosphate, octacalcium phosphate (OCP), brushite (as precursor of HA), and calcium carbonate. Since the interface layer of the implant and the bone is preferably relatively elastic, one or more nano-coatings can optionally be applied. In one exemplary embodiment, at least a part of the at least one applied additive is incorporated in substantially shielded manner in the jacket structure, wherein the additive can be released by means of electromagnetic radiation. In the case that no, or at least insufficient, bone (in)growth takes place, a dosage of bone growth-stimulating substance can, in this manner, be released relatively easily by merely irradiating the implant, whereby surgical intervention can be dispensed with. In addition to irradiation of the implant by means of electromagnetic radiation, it is also possible to envisage causing the implant to vibrate in order to release the additive.

The present disclosure also relates to a method for manufacturing an implant having a substantially solid basic structure and a porous jacket structure at least partially enclosing the basic structure for attachment of cellular tissue, wherein the basic structure and the jacket structure are connected integrally to each other and the porous jacket structure is formed substantially by a structure with open pores, comprising the steps of a) arranging at least one foam-forming mould in an implant-forming mould; b) arranging, in particular casting, a biocompatible material in the implant-forming mould and the foam-forming mould accommodated therein; and c) removing the foam-forming mould from the implant formed during step b). The arranging of the foam-forming mould in the implant-forming mould must take place meticulously and can be realized by means of known techniques. The foam-forming mould will generally be formed here by a mass in which a conduit system is formed to enable forming of the jacket structure. It is also possible to envisage a plurality of foam-forming moulds being arranged simultaneously in the implant-forming mould. In one exemplary embodiment, the arranging, in particular casting, of the biocompatible material in the implant-forming mould as according to step b) takes place at increased temperature. At this increased temperature, the biocompatible material, which is solid at room temperature, will be liquid whereby the material can be cast in both moulds. The method more preferably comprises step d) consisting of allowing the biocompatible material cast in the implant-forming mould to solidify following the arranging, in particular casting, of the biocompatible material in the implant-forming mould as according to step b). Allowing the biocompatible material to solidify generally takes place by allowing the formed implant to cool either actively or passively. The method according to the present disclosure is particularly suitable for producing an implant manufactured from metal or a metal alloy.

In one exemplary embodiment, the method also comprises step e) comprising of optimizing the design of the foam-forming mould before arranging of the at least one foam-forming mould in an implant-forming mould in order to enable the bone growth capacity of the implant for forming to be maximized for a determined application. The manufacture of a foam-forming mould can be described as follows. Firstly, a reticulated foam is placed in a housing (step 1). The foam is then fully infiltrated by means of a heat-resistant material (step 2). The heat-resistant material is subsequently strengthened (step 3) in order to be able to generate a solid structure of the heat-resistant material. The foam with the strengthened, heat-resistant structure therein is further taken out of the housing (step 4), whereafter the foam is removed from the heat-resistant structure (step 5) while forming the actual foam-forming mould which can be applied in the method according to the present disclosure. The removal of the foam can also take place simultaneously with the casting of liquid metal. In the latter case, the foam disappears due to the high temperature of the liquid metal. An alternative would consist of filling a heat-resistant housing with heat-resistant grains (step 1), whereafter a relatively dense packing of the grains can be obtained by means of vibration and pressing (step 2). In general, however, this alternative foam-forming mould will be less preferred as this foam-forming mould is less stable after removal of the housing.

The method may optionally also comprise a step f) finishing the formed implant following the removal of the foam-forming mould from the formed implant as according to step c). The finishing is particularly advantageous in being able to optimize the fit of the implant relative to the bone. The finishing will generally be of a mechanical nature, wherein the implant can, for instance, be finished by means of grinding, sanding and/or polishing after the manufacture thereof.

### BRIEF DESCRIPTION OF THE DRAWINGS

Various aspects of the present disclosure are described hereinbelow with reference to the accompanying FIGURE.

FIG. 1 is a schematic cross-section of an implant according to the present disclosure as a component of a human hip joint.

### DETAILED DESCRIPTION

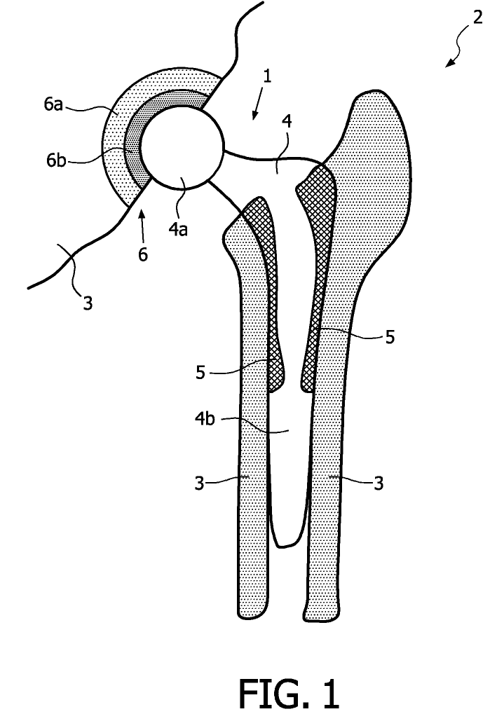

FIG. 1 shows a schematic cross-section of an implant 1 according to the one aspect of the present disclosure as a component of a human hip joint 2. Implant 1, usually also referred to as a prosthesis, is an uncemented implant 1 wherein implant 1 can be anchored to bones 3 forming part of hip joint 2 by means of bone (in)growth. Implant 1 according to the present disclosure comprises for this purpose a substantially solid core 4, which core is, in fact, constructed from a head 4a and a support part 4b connected to head 4a wherein support part 4b is locally enclosed by a porous jacket structure 5. Jacket structure 5 has a net-like structure of mutually connected pores. What is special here is that core 4 and jacket structure 5 are connected integrally to each other, so without intervening adhesive layer, and above all have substantially the same material composition, whereby implant 1 is relatively strong and therefore durable. Due to this particular construction, it is possible for determined properties, such as, for instance, stiffness and design, of both core 4 and jacket structure 5 to be optimized independently of each other whereby the user-friendliness and bone (in)growth, and therefore anchoring to adjacent bones 3, can likewise be optimized. The interface stresses between implant 1 and bones 3 can hereby be minimized. A socket 6 of implant 1 connected to upper bone 3 is also formed by a porous jacket structure. In the shown exemplary embodiment, the core 4 and both jacket structures 5, 6 are manufactured from a biocompatible material, in particular, a metal alloy, and more particularly from a cobalt chrome alloy. Application of a cobalt chrome alloy is advantageous here as this alloy can be brought relatively easily into a state where it can be cast whereby implant 1 can be manufactured in a single production step by means of casting. The porosity of jacket structures 5, 6 is not uniform but progresses gradually in the thickness direction of the respective jacket structure 5, 6. The porosity of each jacket structure 5, 6 close to core 4 preferably lies between 50% and 70%, and between about 85% and 96% close to bone 3, in order, on the one hand, to be able to guarantee sufficient strength and elasticity (plastic deformability) of implant 1 and, on the other hand, to enable optimal bone (in)growth. The number of pores per inch (ppi) of jacket structures 5, 6 is preferably substantially constant and lies between 30 and 45 ppi. As shown in jacket structure 6, which co-acts with head 4a of implant 1, the bone ingrowth will remain limited to only a surface layer 6a of jacket structure 6, wherein a deeper-lying layer 6b of jacket structure 6 will not be (directly) utilized for anchoring of implant 1 to bone 3. Due, however, to the permanent empty pores in this deeper-lying layer 6b of jacket structure 6, a certain permanent elasticity occurs, and thereby a permanent shock-absorbing capacity. Jacket structure 6 can optionally be provided with additives, such as, for instance, bone growth-stimulating agents. These additives are arranged particularly in the pores of surface layer 6a of jacket structure 6, but can also be arranged in the deeper-lying layer 6b of jacket structure 6. In this latter embodiment, it is possible to envisage the additives incorporated in the deeper-lying layer 6b being physically and/or chemically shielded, and it only being possible to release them, if necessary, by means of irradiating the implant 1.

It will be apparent that the present disclosure is not limited to the exemplary embodiments shown and described here, but that numerous variants, which will be self-evident to the skilled person in this field, are possible within the scope of the appended claims.

1. An implant, comprising:
a) a substantially solid basic structure; and
b) a porous jacket structure at least partially enclosing the basic structure for attachment of cellular tissue,
wherein the basic structure and the jacket structure are connected integrally to each other and the jacket structure is formed substantially by a structure with open pores, the porosity of the open ore structure having a gradual progression as seen in the thickness direction.

2. The implant of claim 1, wherein the material composition of the basic structure and the jacket structure are substantially similar.

3. The implant of claim 1, wherein the jacket structure has a substantially plastically deformable structure.

4. The implant of claim 1, wherein the implant is manufactured at least partially from at least one material selected from the group consisting of a biocompatible metal, a biocompatible ceramic, a biocompatible plastic and a biocompatible material with a glass-like structure.

5. (canceled)

6. The implant of claim 1, wherein a part of the jacket structure remote from the basic structure has a porosity similar to that of porous bone.

7. The implant of claim 1, wherein the number of pores per inch (ppi) in the jacket structure is substantially greater than 10 ppi.

8. The implant of claim 1, wherein the pore size of the pores of the jacket structure is substantially between 100 and 1500 μm.

9. The implant of claim 1, wherein the thickness of the jacket structure is substantially between 300 μm and 15 mm.

10. The implant of claim 1, wherein the jacket structure is provided with at least one of additive selected from the group consisting of bone growth-stimulating agents, angiogenesis-stimulating factors, antibacterial agents and inflammation inhibitors.

11. The implant of claim 10, wherein at least a part of the at least one applied additive is incorporated in a substantially shielded manner in the jacket structure, wherein the additive can be released either by means of electromagnetic radiation or by causing the implant to vibrate.

12. (canceled)

13. (canceled)

14. (canceled)

15. (canceled)

16. (canceled)

17. The implant of claim 2, wherein the jacket structure has a substantially plastically deformable structure.
